(19) United States
(12) Patent Application Publication
(10) Pub. No.: US 2026/0114394 A1
(43) Pub. Date: Apr. 30, 2026

(54) PROPOGATION METHOD USING CLONAL SEEDS

(71) Applicant: Rev Genomics, Inc., Oakland, CA (US)

(72) Inventors: Phillip Adam Conklin, Oakland, CA (US); Bertrand R. Vick, Oakland, CA (US)

(73) Assignee: Rev Genomics, Inc., Oakland, CA (US)

(21) Appl. No.: 19/256,126

(22) Filed: Jun. 30, 2025

Related U.S. Application Data
(60) Provisional application No. 63/666,049, filed on Jun. 28, 2024.

(51) Int. Cl. A01H 1/02; A01H 6/28

(52) U.S. Cl. CPC A01H 1/023; A01H 1/026; A01H 6/28

(57) ABSTRACT

Exemplary embodiments described herein include various methods for creating clonal seeds from which cannabis females are generated. Clonal seeds are cannabis seeds that give rise to identical plants. Specifically, clonal seeds may be genetically identical in certain embodiments and almost genetically identical in other embodiments.

### CROSS REFERENCE TO RELATED PATENT APPLICATIONS

The present application claims priority benefit, under 35 U.S.C. § 119(e), to co-pending and commonly-assigned U.S. Provisional Patent Application No. 63/666,049, filed Jun. 28, 2024 entitled “CLONAL SEEDS AS SUPERIOR METHOD FOR PROPAGATION OF CANNABIS WITH HIGHER YIELDS THAN CLONES”, and listing as inventors Bertrand R. V

### BACKGROUND

One skilled in the art will recognize the increasing growth and applicability of cannabis-based products within today's society and health markets. For example, the use of medical cannabis has been known to treat a variety of medical conditions including anxiety, sleep disorder and other types of conditions. This expanding use of medical cannabis products has resulted in a variety of treatments that are oftentimes more effective with less side-effects than previously used traditional medicines. In addition, recreational cannabis use is drastically increasing as state laws have become more tolerant in its use. This increasing demand has led to a need to more efficiently and dynamically develop growth techniques within the cannabis markets.

Cannabis plants may be differentiated across three types: namely female cannabis, male cannabis and hermaphrodite cannabis. In most instances, the female cannabis plant is preferred across a variety of different cannabis uses and applications. However, maximizing female cannabis plants across a cannabis crop presents certain challenges and failure to address these challenges may result in a meaningful amount of male or hermaphrodite cannabis plants being present. One factor in the value of a cannabis crop is the ratio of female cannabis plants to male/hermaphrodite plants in that a higher density of female cannabis results in an increase in value and applicability across different uses.

Accordingly, it is desired that systems and methods be provided that increase the amount of female cannabis plants within a crop.

### BRIEF DESCRIPTION OF THE DRAWINGS

The accompanying drawings, where like reference numerals refer to identical or functionally similar elements throughout the separate views, together with the detailed description below, are incorporated in and form part of the specification, and serve to further illustrate embodiments of concepts that include the claimed disclosure and explain various principles and advantages of those embodiments.

The methods and systems disclosed herein have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

FIG. 1 shows exemplary methods for creating plants with clonal seeds according to various embodiments of the invention.

FIG. 2 shows an exemplary method for using inbreeding to create mostly homozygous lines according to various embodiments of the invention.

FIG. 3 shows exemplary growth and yields of plants from clonal seeds versus clones according to various embodiments of the invention.

FIG. 4 shows an alternative view of exemplary growth and yields of plants from clonal seeds versus clones according to various embodiments of the invention.

### DETAILED DESCRIPTION

In the following description, for purposes of explanation, specific details are set forth in order to provide an understanding of the disclosure. It will be apparent, however, to one skilled in the art that the disclosure can be practiced without these details. Furthermore, one skilled in the art will recognize that embodiments of the present disclosure, described below, may be implemented in a variety of ways, such as a process, a plant or plant structure, or a method of propagation.

Reference in the specification to “one or more embodiments,” “preferred embodiment,” “an embodiment,” “embodiments,” or the like means that a particular feature, structure, characteristic, or function described in connection with the embodiment is included in at least one embodiment of the disclosure and may be in more than one embodiment. Also, the appearances of the above-noted phrases in various places in the specification are not necessarily all referring to the same embodiment or embodiments.

The use of certain terms in various places in the specification is for illustration and should not be construed as limiting. The terms “include,” “including,” “comprise,” and “comprising” shall be understood to be open terms and any examples are provided by way of illustration and shall not be used to limit the scope of this disclosure.

A service, function, or resource is not limited to a single service, function, or resource; usage of these terms may refer to a grouping of related services, functions, or resources, which may be distributed or aggregated. The words “optimal,” “optimize,” “optimization,” and the like refer to an improvement of an outcome or a process and do not require that the specified outcome or process has achieved an “optimal” or peak state.

It shall be noted that: (1) certain steps may optionally be performed; (2) steps may not be limited to the specific order set forth herein; (3) certain steps may be performed in different orders; and (4) certain steps may be done concurrently.

Cannabis (both hemp and “marijuana”) is typically dioecious, meaning that male and female reproductive organs exist on separate individuals (although there are rare, but important, exceptions to this rule). Thus, when cannabis seeds are created by crossing a male to a female, statistically half the seeds will create male plants, and half the seeds will create female plants, which is undesirable for many cannabis use cases, as it is the female plants that produce valuable secondary metabolites (THC, CBD, etc.). It is possible to produce “feminized” seeds that will give rise to mostly female plants by coaxing, using primarily chemical methods, a female cannabis plant to produce male reproductive parts and pollen. Since cannabis uses an X-Y sex-determination system like humans, genetically female plants can make XX pollen using these chemical feminization methods.

Despite the fact that such feminized seeds can be created, seeds are not the de facto propagation method in the various sectors of the cannabis industry (industrial, medical, recreational) for the simple fact that almost all cannabis varietals are highly hybridized. When these highly hybridized varieties are crossed, genetic segregation occurs, and the progeny will be genotypically and phenotypically diverse—this is a very negative characteristic when a monocrop is desired. As a result, much (or most) cannabis is propagated by creating clones from characterized plants. The cloning process (described elsewhere) is laborious and expensive, and clones results in adult cannabis plants that are far inferior to cannabis plants that come from seeds. For example, many may often carry diseases and may be more expensive to produce or purchase than seeds. Unfortunately, the cannabis industry has been forced to use clones and cloning because cannabis seeds confer little-to-no consistency in the resulting plants.

The creation of clonal seeds is an extraordinary moment for the development of cannabis into an industrially-relevant crop. Clonal seeds are cannabis seeds that give rise to identical plants. Specifically, clonal seeds may be genetically identical in certain embodiments and almost genetically identical in other embodiments. These clonal seeds have never existed before and present a variety of advantages and benefits for an ever-growing and complicated cannabis industry.

In certain embodiments, clonal seeds are made by creating perfectly inbred or near perfectly inbred plants. These inbred plant parents are subsequently mated and the offspring are identical to one another. The plants that are directly generated from clonal seeds have higher yields and higher THC and terpenoid content than even clones from seed plants. In other words, these plants from clonal seeds have better performance than even cut-clones if the exact same genetic background.

Clones, which have been the dominant propagation starting material, are expensive, are often riddled with diseases and pests like mites and Hop Latent Viroid (HLVd), and require “mother” plants for their production (which require a large amount of space and labor for their housing and maintenance). Clonal seeds will completely obviate the use of clones or substantially reduce the use of clones, as they mitigate all the problems of clones while capturing all the benefits thereof (of which genetic consistency is the primary benefit).

Others have developed seeds that achieve some of what clonal seeds offer, but none of the other efforts have been able to achieve the genetic consistency of clonal seeds. For instance, cannabis breeders will self-fertilize cultivars, using the feminization process described above, for several generations to increase the homozygosity of the cannabis genome. One round of selfing will roughly cut the heterozygous loci in half, resulting in more consistent progeny with each subsequent round of selfing. However, if you only achieve 50% reduction of heterozygous loci with each round of selfing, you only approach and never reach 100% homozygosity. Depending on how homozygous a cultivar already is, a few rounds of selfing is usually insufficient to produce the consistency needed for reliable mono-cropping. Furthermore, because cannabis evolved as a dioecious, out-crossing species, each round of selfing often results in inbreeding depression.

According to various exemplary embodiments provided herein, clonal seeds are created using methods disclosed herein that provide the advantages mentioned above. For example, these seeds provide consistency and give rise to plants that are genetically and phenotypically identical. Thus, clonal seeds provide many of the benefits of seeds with the consistency of clones. Furthermore, the plants from these seeds have higher yields than clones from the plants. In other words, using the exact same genetic background, the clonal seeds have superior yields to clones.

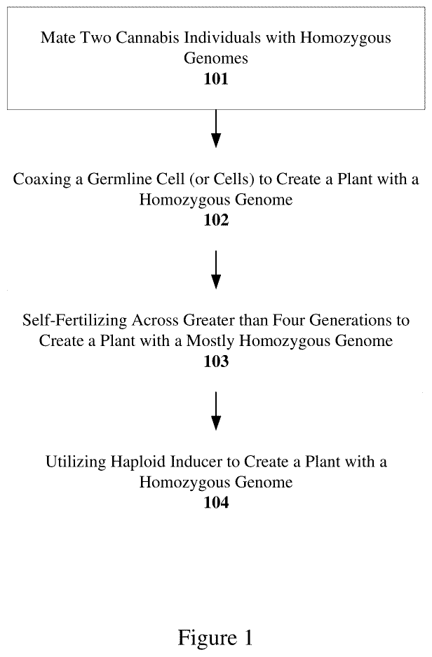

FIG. 1 shows exemplary methods for creating plants with clonal seeds according to various embodiments of the invention.

A cannabis seed, called a clonal seed, gives rise to genetically identical offspring or near genetically identical offspring that have higher yields than cannabis plants derived from clones. In various exemplary embodiments 101, two cannabis individuals with homozygous genomes are mated to produce seeds that give rise to plants with superior yields to clones.

In some exemplary embodiments 102, a cannabis plant with a homozygous genome is created by coaxing a germline cell (or cells) to develop into a plant with roots, stems and shoots.

In certain exemplary embodiments 103, a cannabis plant with a homozygous genome is created by self-fertilizing across greater than 4 generations.

In other exemplary embodiments 104, a cannabis plant with a homozygous genome is created by utilizing haploid inducer lines.

In various embodiments, a first step in the production of clonal seeds of cannabis, according to many exemplary embodiments, is to start with parents each with a homozygous genome. This can be accomplished in multiple ways. The concept here is that germline cells, which are 1x (as opposed to 2x, as is the case with somatic cells), could be isolated and encouraged to double their genome. This process of doubling the genome would lead to normal diploid plants where each chromosome is identical to its homologous chromosome, hence the genome would be effectively homozygous. Alternatively, 1x plants (instead of just cells) could be developed from the germline, then microtubule depolymerization inhibitors (such as colchicine or orxyalin) could be utilized after the fact to potentiate chromosome doubling. And yet another method to make the cannabis genome mostly homozygous is 8-9 rounds of self-fertilization. One skilled in the art will recognize that a variety of rounds may be implemented across different embodiments of the invention. In certain examples, self-fertilization processes may never result in a perfectly homozygous genome, but it could potentially come close. CENH3 mutants may also be employed effectively to this end of creating plants with 100% homozygous genomes (Ravi, Maruthachalam, and Simon WL Chan. “Haploid plants produced by centromere-mediated genome elimination.” Nature 464.7288 (2010): 615-618.).

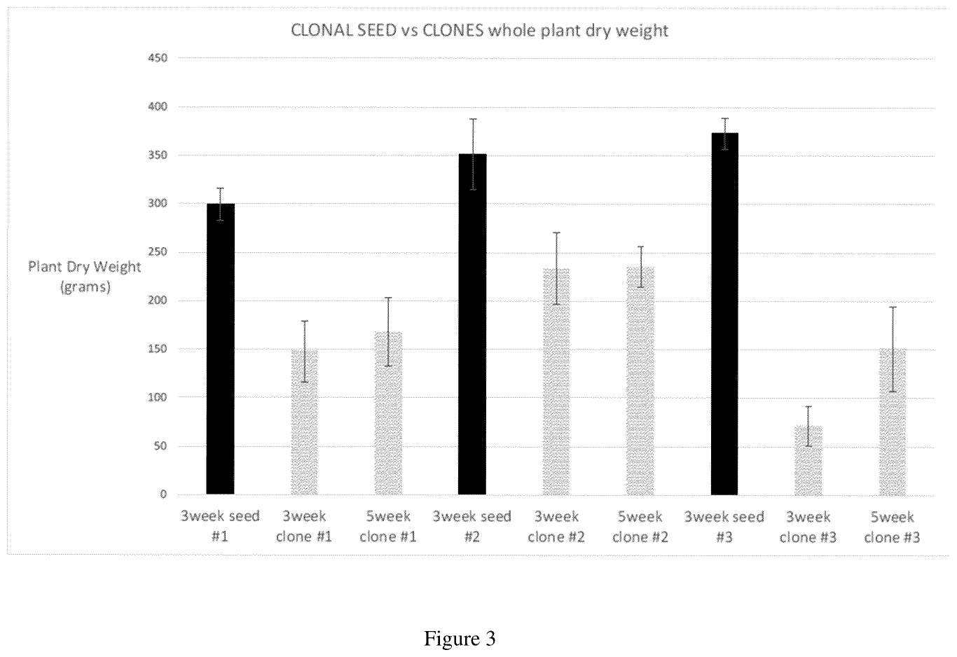

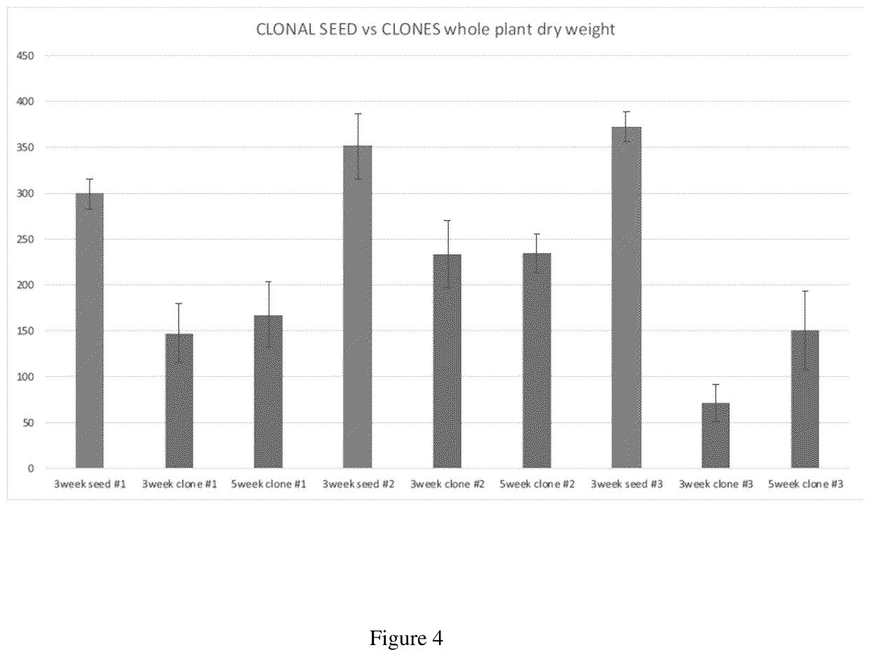

According to various embodiments, cultivars are mated at this point such that a mated cultivar possess completely homozygous genomes in order to produce clonal seeds. Additionally, the corresponding yields of clones (taken from clonal seed plants) versus clonal seed plants themselves, an experiment that was never possible given that genetically identical plants from seed never existed before. An astounding difference is identified between the growth and yields of plants from clonal seeds versus clones, i.e., see FIGS. 3-4.

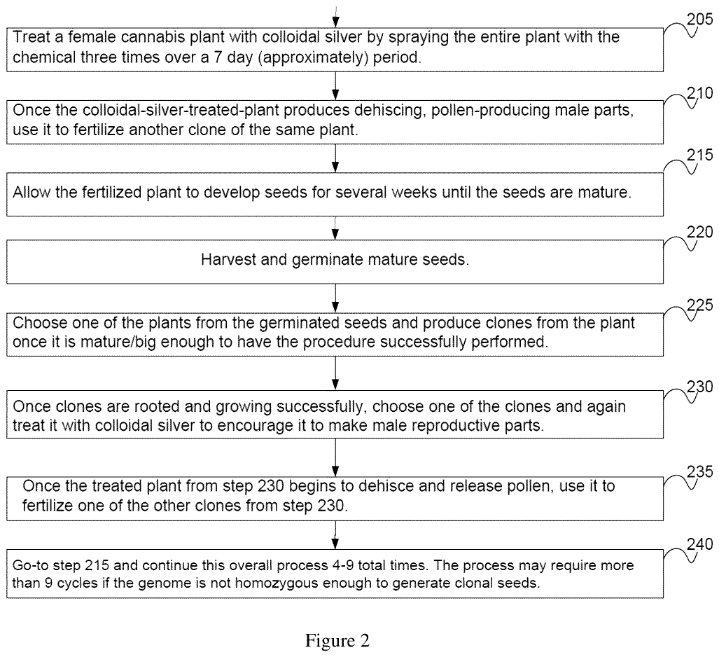

FIG. 2 shows an exemplary method 200 for using inbreeding to create mostly homozygous lines according to various embodiments of the invention.

At step 205, treat a female cannabis plant with colloidal silver by spraying the entire plant with the chemical three times over a 7 day (approximately) period. One skilled in the art will recognize that the number of times colloidal silver is applied and the time over which the application occurs may vary across different embodiments.

At step 210, once the colloidal-silver-treated-plant produces dehiscing, pollen-producing male parts, use the dehiscing, pollen-producing male parts to fertilize another clone of the same plant.

At step 215, allow the fertilized plant to develop seeds for several weeks until the seeds are mature. One skilled in the art will recognize that time used for developing seeds may vary across a different embodiments of the invention.

At step 220, the mature seeds are harvested and germinated.

At step 225, plants from the germinated seeds are selected and used to produce clones from the plant once it is mature/big enough to have the procedure successfully performed. In certain embodiments, the maturity of the plant relates to the success of the clone. A series of experiments may be performed that correlates the quality of clones to the maturity of the plants. One skilled in the art will recognize that various embodiments of the invention cover different maturity levels of the plants.

At step 230, once clones are rooted and growing successfully, choose one of the clones and again treat it with colloidal silver to encourage it to make male reproductive parts.

At step 235, once the treated plant from step 230 begins to dehisce and release pollen, use it to fertilize one of the other clones from step 230.

At step 240, go-to step 215 and continue this overall process at least 4 times. In certain embodiments, the process may require more than 9 cycles if the genome is not homozygous enough to generate clonal seeds. One skilled in the art will recognize that various embodiments of the invention cover all repetitions of this process beyond four times.

FIG. 3 shows exemplary growth and yields of plants from clonal seeds versus clones in various embodiments of the invention.

Yields of plants from clonal seeds (black) were compared to clones taken from clonal seed plants (grey), such that the exact same genetic backgrounds are being compared. As shown, three independent cultivars are compared in triplicate in accordance to various embodiments of the invention. Clonal seeds were grown vegetatively for 3 weeks and compared to clones that were grown vegetatively for 3 weeks or 5 weeks. However, one skilled in the art will recognize that the time in which the clones were grown may vary across different embodiments of the in invention.

After vegetative growth, plants were transitioned to a flower-inducing light cycle and allowed to develop for 10 weeks in certain embodiments. In one particular example, the yields are reported in grams after drying the plants for 4 weeks at 60 F and 60% relative humidity, then storage in plastic bags for another 4 weeks. One skilled in the art will recognize that the time durations of both drying and storage may vary across different embodiments. Furthermore, the temperature and humidity levels may also vary across different embodiments of the present invention.

FIG. 4 shows an alternative view of exemplary growth and yields of plants from clonal seeds versus clones according to various embodiments of the invention. One skilled in the art will recognize the correlation between yields of clonal seeds versus clones applies across a number of variable values, all of which are intended to fall within the scope of embodiments of the invention.

While various embodiments have been described above, they have been presented by way of example only, and not limitation. The descriptions are not intended to limit the scope of the invention to the forms set forth herein. To the contrary, the present descriptions are intended to cover such alternatives, modifications, and equivalents as may be included within the spirit and scope of the invention as defined by the appended claims and otherwise appreciated by one of ordinary skill in the art. Thus, the breadth and scope of a preferred embodiment should not be limited by any of the above-described exemplary embodiments

What is claimed is:

1. A method for propagating clonal seeds, the method comprising:
treat a first female cannabis plant with colloidal silver;
after the colloidal-silver-treated cannabis plant produces pollen-producing male parts, use the pollen producing male parts to fertilize a clone of the first female cannabis plant;
identify at least one mature seed on the fertilized clone;
harvest and germinate the at least one mature seed such that at least one second cannabis plant is produced from the germinated at least one mature seed;
select a preferred cannabis plant from the at least one second cannabis plant; and
treat the at least one second cannabis plant with colloidal silver to enable subsequent production of clonal seeds.

2. The method for propagating clonal seeds of claim 1 wherein the first female cannabis plant is treated with colloidal silver at least three times.

3. The method of claim 2 wherein the first female cannabis plant is treated with colloidal silver for at least seven days.

4. The method of claim 2 wherein the first female cannabis plant is treated at least three times with each treatment being separated by a preferred time period.

5. The method of claim 1 wherein the pollen-producing male parts are produced by a dehiscing process.

6. The method of claim 1 wherein the at least one mature seed is identified based on a maturity threshold.

7. The method of claim 1 wherein the at least one mature seed is identified based on a size threshold.

8. The method of claim 1 wherein the steps are performed at least four times.

9. The method of claim 8 wherein the steps are performed less than ten times.
